(19) United States
(12) Patent Application Publication
(10) Pub. No.: US 2009/0043389 A1
(43) Pub. Date: Feb. 12, 2009

(54) CARTILAGE IMPLANT PLUG WITH FIBRIN GLUE AND METHOD FOR IMPLANTATION

(76) Inventors: GORDANA VUNJAK-NOVAKOVIC, New York, NY (US); Arthur A. Gertzman, Flemington, NJ (US); Moon Hae Sunwoo, Old Tappan, NJ (US); William W. Tomford, Belmont, NJ (US); Katherine G. Truncale, Hillsborough, NJ (US)

Correspondence Address: GREENBERG TRAURIG, LLP, 200 PARK AVE., P.O. BOX 677, FLORHAM PARK, NJ, 07932, US

(21) Appl. No.: 12/191,490

(22) Filed: Aug. 14, 2008

Related U.S. Application Data
(62) Division of application No. 10/815,778, filed on Apr. 2, 2004.

(51) Int. Cl. A61F 2/08

(52) U.S. Cl. 623/14.12

(57) ABSTRACT

The invention is directed toward a cartilage repair assembly comprising a shaped structure of subchondral bone with an integral overlying cartilage cap which is treated to remove cellular debris and proteoglycans and milled cartilage in a bioabsorbable carrier. The shaped structure is dimensioned to fit in a drilled bore in a cartilage defect area so that said shaped bone and cartilage cap when centered in the bore does not engage the side wall of the bore in an interference fit and is surrounded by milled cartilage and carrier. A method for inserting the assembly into a cartilage defect area is disclosed.

### RELATED APPLICATIONS

This is a divisional application of U.S. patent application Ser. No. 10/815,778 filed Apr. 2, 2004, which is incorporated by reference herein in is entirety.

### FIELD OF INVENTION

The present invention is generally directed toward a surgical implant and is more specifically directed toward an implant for a joint having a cartilage face and bone body for implantation in a shoulder, hip, elbow, ankle, knee or temporomandibular joint.

### BACKGROUND OF THE INVENTION

Articular cartilage injury and degeneration present medical problems to the general population which are constantly addressed by the orthopedic surgeon. Every year in the United States, over 500,000 arthroplastic or joint repair procedures are performed. These include approximately 125,000 total hip and 150,000 total knee arthroplastics and over 41,000 open and arthroscopic procedures to repair cartilaginous defects of the knee. Chen et al. “Repair of Articular Cartilage Defects: Part 1, Basic Science of Cartilage Healing, American Journal of Orthopaedics 1999, January 31-33.

In the knee joint the articular cartilage tissue forms a lining which faces the joint cavity on one side and is linked to the subchondral bone plate by a narrow layer of calcified cartilage tissue on the other. Articular cartilage (hyaline cartilage) consists primarily of extracellular matrix with a sparse population of chondrocytes distributed throughout the tissue. Articular cartilage is composed of chondrocytes, type II collagen fibril network, proteoglycans and water. Active chondrocytes are unique in that they have a relatively low turnover rate and are sparsely distributed within the surrounding matrix. The collagens give the tissue its form and tensile strength and the interaction of proteoglycans with water give the tissue its stiffness for compression, resilience and durability. The hyaline cartilage provides a low friction bearing surface over the bony parts of the joint. If the lining becomes worn or damaged resulting in lesions, joint movement may be painful or severely restricted. Whereas damaged bone typically can regenerate successfully, hyaline cartilage regeneration is quite limited because of its limited regeneration and reparative abilities.

Articular cartilage lesions generally do not heal, or heal only partially under certain biological conditions due to the lack of nerves, blood vessels and a lymphatic system. The limited reparative capabilities of hyaline cartilage generally results in the generation of repair tissue that lacks the structure and biomechanical properties of normal cartilage. Generally, the healing of the defect results in a fibrocartilaginous repair tissue that lacks the structure and biomedical properties of hyaline cartilage and degrades over the course of time. Articular cartilage lesions are frequently associated with disability and with symptoms such as joint pain, locking phenomena and reduced or disturbed function. These lesions are difficult to treat because of the distinctive structure and function of hyaline cartilage. Such lesions are believed to progress to severe forms of osteoarthritis. Osteoarthritis is the leading cause of disability and impairment in middle-aged and older individuals, entailing significant economic, social and psychological costs. Each year, osteoarthritis accounts for as many as 39 million physician visits and more than 500,000 hospitalizations. By the year 2020, arthritis is expected to affect almost 60 million persons in the United States and to limit the activity of 11.6 million persons. Jackson et al., “Cartilage Substitute, Overview of Basic Science and Treatment Options”, Journal of American Academy of Orthopedic Surgeons, 2001, 9:37-52.

There are many current therapeutic methods being used. None of these therapies has resulted in the successful regeneration of hyaline-like tissue that withstands normal joint loading and activity over prolonged periods. Currently, the techniques most widely utilized clinically for cartilage defects and degeneration are not articular cartilage substitution procedures, but rather lavage, arthroscopic debridement, and repair stimulation. The direct transplantation of cells or tissue into a defect and the replacement of the defect with biologic or synthetic substitutions presently accounts for only a small percentage of surgical interventions. The optimum surgical goal is to replace the defects with cartilage-like substitutes so as to provide pain relief reduce effusions and inflammation, restore function, reduce disability and postpone or alleviate the need for prosthetic replacement.

Lavage and arthroscopic debridement involve irrigation of the joint with solutions of sodium chloride, Ringer or Ringer and lactate. The temporary pain relief is believed to result from removing degenerative cartilage debris, proteolytic enzymes and inflammatory mediators. These techniques provide temporary pain relief, but have little or no potential for further healing.

Repair stimulation is conducted by means of drilling, abrasion arthroplasty or microfracture. Penetration into the subchondral bone induces bleeding and fibrin clot formation which promotes initial repair, however, the tissue formed is fibrous in nature and not durable. Pain relief is temporary as the tissue exhibits degeneration, loss of resilience, stiffness and wear characteristics over time.

The periosteum and perichondrium have been shown to contain mesenchymal progenitor cells capable of differentiation and proliferation. They have been used as grafts in both animal and human models to repair articular defects. Few patients over 40 years of age have obtained good clinical results, which most likely reflects the decreasing population of osteochondral progenitor cells with increasing age. There have also been problems with adhesion and stability of the grafts, which result in their displacement or loss from the repair site.

Transplantation of cells grown in culture provides another method of introducing a new cell population into chondral and osteochondral defects. Carticel® is a commercial process to culture a patient's own cartilage cells for use in the repair of cartilage defects in the femoral condyle and is marketed by Genzyme Biosurgery in the United States and Europe. The procedure uses arthroscopy to take a biopsy from a healthy, less loaded area of knee articular cartilage. Enzymatic digestion of the harvested tissue releases the cells that are sent to a laboratory where they are grown for a period ranging from 2-5 weeks. Once cultivated, the autologous cells are injected during a more open and extensive knee procedure into areas of defective cartilage where it is hoped that they will facilitate the repair of damaged tissue. An autologous periosteal flap with cambium layer is used to seal the transplanted cells in place and act as mechanical barrier. Fibrin glue is used to seal the edges of the flap. Proponents of this procedure report that it produces satisfactory results, including the ability to return to demanding physical activities, in more than 80% of patients and that biopsy specimen of the tissue in the graft sits show hyaline-like cartilage repair. However, long term studies of this procedure in rabbits and dogs showed limited success and showed degradation at the implant site. The original study report has been criticized for not being a prospective controlled randomized study and for lack of quantitative or mechanical. Of interest, a 14 year follow-up of a similar patient group that underwent diagnostic arthroscopy in combination with one of several treatments (removal of bone bodies, shaving, Pride drilling) had good to excellent knee function in 78% of the patients. Thus, further studies are needed to assess the function and durability of the new tissue to determine whether it improves joint function and delays or prevents joint degeneration.

As with the perichondrial graft, patient/donor age may compromise the success of this procedure as chondrocyte population decreases with increasing age. Disadvantages to this procedure include the need for two separate surgical procedures, potential damage to surrounding cartilage when the periosteal patch is sutured in place, the requirement of demanding complex microsurgical techniques, and the expensive cost of the procedure which is currently not covered by insurance.

Osteochondral transplantation or mosaicplasty involves excising all injured or unstable tissue from the articular defect and creating cylindrical holes in the base of the defect and underlying bone. These holes are filled with autologous cylindrical plugs of healthy cartilage and bone in a mosaic fashion. The osteochondral plugs are harvested from a lower weight-bearing area of lesser importance in the same joint. This technique, shown in Prior Art FIG. 2, can be performed as arthroscopic or open procedures. Reports of results of osteochondral plug autografts in a small numbers of patients indicate that they decrease pain and improve joint function. Factors that can compromise the results include donor site morbidity, effects of joint incongruity on the opposing surface of the donor site, damage to the chondrocytes at the articular margins of the donor and recipient sites during preparation and implantation, and collapse or settling of the graft over time. The limited availability of sites for harvest of osteochondral autografts restricts the use of this approach to treatment of relatively small articular defects and the healing of the chondral portion of the autograft to the adjacent articular cartilage remains a concern.

Transplantation of large allografts of bone and overlying articular cartilage is another treatment option that involves a greater area than is suitable for autologous cylindrical plugs, as well as for a non-contained defect. The advantages of osteochondral allografts are the potential to restore the anatomic contour of the joint, lack of morbidity related to graft harvesting, greater availability than autografts and the ability to prepare allografts in any size to reconstruct large defects. Clinical experience with fresh and frozen osteochondral allografts shows that these grafts can decrease joint pain, and that the osseous part of an allograft can heal to the host bone and the chondral part can function as an articular surface. Drawbacks associated with this methodology in the clinical situation include the scarcity of fresh donor material and problems connected with the handling and storage of frozen tissue. Fresh allografts carry the risk of immune response or disease transmission. Musculoskeletal Transplant Foundation (MTF) has preserved fresh allografts in a media that maintains a cell viability of 50% for 35 days for use as implants. Frozen allografts lack cell viability and have shown a decreased amount of proteoglycan content which contribute to deterioration of the tissue.

A number of United States patents have been specifically directed towards bone plugs which are implanted into a bone defect. Examples of such bone plugs are U.S. Pat. No. 4,950,296 issued Aug. 21, 1990 which discloses a bone graft device comprising a cortical shell having a selected outer shape and a cavity formed therein for receiving a cancellous plug, and a cancerous plug-fitted into the cavity in a manner to expose at least one surface; U.S. Pat. No. 6,039,762 issued Mar. 21, 2000 having a cylindrical shell with an interior body of deactivated bone material and U.S. Pat. No. 6,398,811 issued Jun. 4, 2002 directed to a bone spacer which has a cylindrical cortical bone plug with an internal throughgoing bore designed to hold a reinforcing member. U.S. Pat. No. 6,383,211 issued May 7, 2002 discloses an invertebral implant having a substantially cylindrical body with a throughgoing bore dimensioned to receive bone growth materials.

U.S. Pat. No. 6,379,385 issued Apr. 30, 2002 discloses an implant base body of spongious bone material into which a load carrying support element is embedded. The support element can take the shape of a diagonal cross or a plurality of cylindrical pins. See also, U.S. Pat. No. 6,294,187 issued Sep. 25, 2001 which is directed to a load bearing osteoimplant made of compressed bone particles in the form of a cylinder. The cylinder is provided with a plurality of throughgoing bores to promote blood flow through the osteoimplant or to hold a dimineralized bone and glycerol paste mixture. U.S. Pat. No. 6,096,081 issued Aug. 1, 2000 shows a bone dowel with a cortical end cap or caps at both ends, a brittle cancerous body and a throughgoing bore.

A number of patents in the prior art show the use of bone putty, pastes or gels to fill bone defects. U.S. Pat. No. 5,290,558 issued Mar. 1, 1994 discloses a flowable demineralized bone powder composition using an osteogenic bone powder with large particle size ranging from about 0:1 to about 1.2 cm. mixed with a low molecular weight polyhydroxy compound possessing from 2 to about 18 carbons including a number of classes of different compounds such as monosaccharides, disaccharides, water dispersible oligosaccharides and polysaccharides.

Another such bone gel is disclosed in the U.S. Pat. No. 5,073,373 issued Dec. 17, 1991. Bone lamellae in the shape of threads or filaments retaining low molecular weight glycerol carrier are disclosed in U.S. Pat. No. 5,314,476 issued May 24, 1994 and U.S. Pat. No. 5,507,813 issued Apr. 16, 1996 and the tissue forms described in these patents are known commercially as the GRAFTON® Putty and Flex, respectively.

U.S. Pat. No. 5,356,629 issued Oct. 18, 1994 discloses making a rigid gel in the nature of a bone cement to fill defects in bone by mixing biocompatible particles, preferably polymethylmethacrylate coated with polyhydroxyethylmethacrylate in a matrix selected from a group which lists hyaluronic acid to obtain a molded semi-solid mass which can be suitably worked for implantation into bone. The hyaluronic acid can also be utilized in monomeric form or in polymeric form preferably having a molecular weight not greater than about one million Daltons. It is noted that the nonbioabsorbable material which can be used to form the biocompatible particles can be derived from xenograft bone, homologous bone, autogenous bone as well as other materials. The bioactive substance can also be an osteogenic agent such as demineralized bone powder, morselzed cancellous bone, aspirated bone marrow and other autogenous bone sources. The average size of the particles employed is preferably about 0.1 to about 3.0 mm, more preferably about 0.2 to about 1.5 mm, and most preferably about 0.3 to about 1.0 mm. It is inferentially mentioned but not taught that particles having average sizes of about 7,000 to 8,000 microns, or even as small as about 100 to 700 microns can be used.

U.S. Pat. No. 4,172,128 issued Oct. 23, 1979 discloses a demineralized bone, material mixed with a carrier to reconstruct tooth or bone material by adding a mucopolysaccharide to a mineralized bone colloidal material. The composition is formed from a demineralized coarsely ground bone materials which may be derived from human bones and teeth, dissolved in a solvent forming a colloidal solution to which is added a physiologically inert polyhydroxy compound such as mucopolysaccharide or polyuronic acid in an amount which causes orientation when hydrogen ions or polyvalent metal ions are added to form a gel. Example 25 of the patent notes that mucopolysaccharides produce pronounced ionotropic effects and that hyaluronic acid is particularly responsible for spatial cross-linking.

U.S. Pat. No. 6,030,635 issued Feb. 29, 2000 and U.S. Pat. No. 6,437,018 issued Aug. 20, 2002 are directed toward a malleable bone putty and a flowable gel composition for application to a bone defect site to promote new bone growth at the site which comprises a new bone growth inducing compound of demineralized lyophilized allograft bone powder. The bone powder has a particle size ranging from about 100 to about 850 microns and is mixed in a high molecular weight hydrogel carrier which contains a sodium phosphate saline buffer.

The use of implants for cartilage defects is much more limited. Aside from the fresh allograft implants and autologous implants, U.S. Pat. No. 6,110,209 issued Nov. 5, 1998 shows the use an autologous articular cartilage cancerous bone paste to fill arthritic defects. The surgical technique is arthroscopic and includes debriding (shaving away loose or fragmented articular cartilage), followed by morselizing the base of the arthritic defect with an awl until bleeding occurs. An osteochondral graft is then harvested from the inner rim of the intercondylar notch using a trephine. The graft is then morselized in a bone graft crusher, mixing the articular cartilage with the cancellous bone. The paste is then pushed into the defect and secured by the adhesive properties of the bleeding bone. The paste can also be mixed with a cartilage stimulating factor, a plurality of cells, or a biological glue. All patients are kept non-weight bearing for four weeks and used a continuous passive motion machine for six hours each night. Histologic appearance of the biopsies have mainly shown a mixture of fibrocartilage with hyaline cartilage. Concerns associated with this method are harvest site morbidity and availability, similar to the mosaicplasty method.

U.S. Pat. No. 6,379,367 issued Apr. 30, 2002 discloses a plug with abase membrane, a control plug, and a top membrane which overlies the surface of the cartilage covering the defective area of the joint.

### SUMMARY OF THE INVENTION

A cartilage allograft construct assembly comprising a plug with a bone base and cartilage cap for treating articular cartilage defects. The plug is used together with a milled cartilage paste which surrounds the plug in a bore which has cut into the patient to remove the lesion area. The process for inserting the construct plug is to arthroscopically remove one or more osteochondral plugs from the defect area. A small amount of biological glue is inserted into the defect and the plug is inserted into the surgically created cylindrical defect. The plug is then positioned so that it is flush and covered with paste or putty. Additives may be applied to the assembly in order to increase chondrocyte migration and proliferation. Stem cells or chondrocytes may also be applied to the construct to restore the matrix. Each allograft construct can support the addition of a variety of chondrogenic stimulating factors including, but not limited to growth factors (FGF-2, FGF-5, IGF-1, TGF-β, BMP-2, BMP-7, PDGF, VEGF), human allogenic or autologous chondrocytes, human allogenic or autologous bone marrow cells, demineralized bone matrix, insulin, insulin-like growth factor-1, transforming growth factor-B, interleukin-1 receptor antagonist, hepatocyte growth factor platelet-derived growth factor, Indian hedgehog and parathyroid hormone-related peptide or bioactive glue.

The implant is placed in a bore or hole cut in the patient to remove the lesion area and the milled cartilage paste is used to fill the space not occupied by the plug.

### BRIEF DESCRIPTION OF THE DRAWINGS

FIG. 1 shows the anatomy of a knee joint;

FIG. 2 shows a schematic mosaicplasty as known in the prior art;

FIG. 3 shows a schematic perspective view of a cylindrical allograft osteochondral plug assembly with a cartilage paste or putty in a defect site;

FIG. 4 shows a perspective view of the osteochondral plug used in FIG. 3;

FIG. 5 shows a perspective view of another embodiment of a oval shaped allograft osteochondral plug assembly with a cartilage paste or putty in a defect site;

FIG. 6 shows a perspective view of the oval osteochondral plug used in FIG. 5;

FIG. 7 shows a schematic perspective view of another embodiment of a scalloped shaped allograft osteochondral assembly with a cartilage paste or putty in a defect site;

FIG. 8 shows a perspective view of a scalloped shaped osteochondral plug used in FIG. 7;

FIG. 9 shows a schematic perspective view of another embodiment of a cruciate shaped allograft osteochondral assembly with a cartilage paste or putty in a defect site; and

FIG. 10 shows a perspective view of a cruciate shaped osteochondral plug used in FIG. 9.

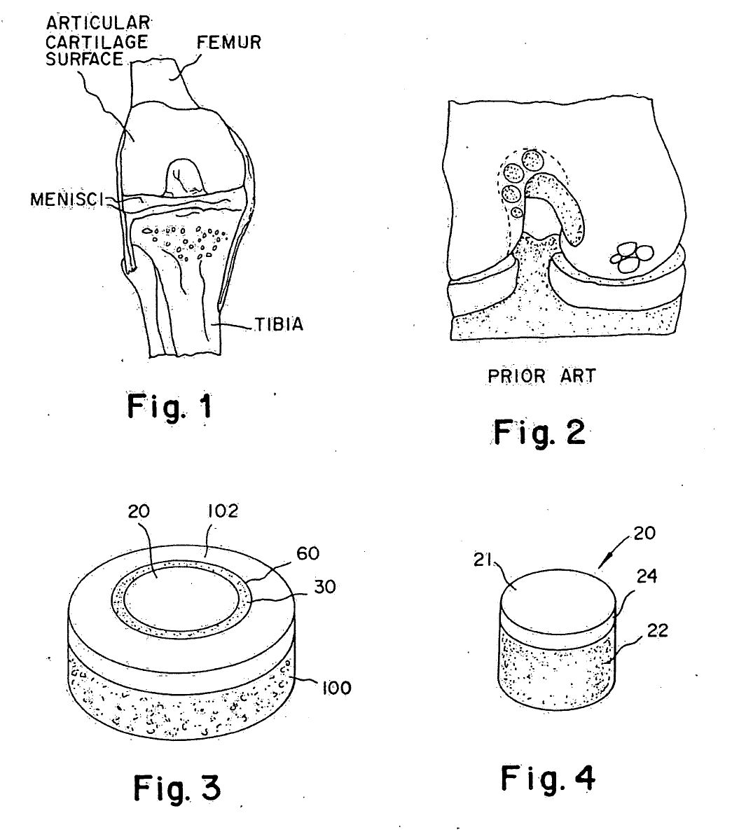

### DESCRIPTION OF THE INVENTION

The present invention is directed towards cartilage repair using an osteochondral plug assembly and method of treatment. The preferred embodiment and best mode of the invention is shown in FIGS. 3 and 4. In the production of the invention, an allograft plug 20 having a subchondral bone body 22 and an overlying cap 24 of hyaline cartilage is treated to remove cellular material, chondrocytes and pluripotent mesenchymal cells and proteoglycans freezing same −20° C. to −80° C., and lyophilized reducing its water content.

In the treatment for cell and proteoglycan extraction the plug 20 was soaked in hyaluronidase (type IV-s, 3 mg/mL), trypsin (0.25% in monodibasic buffer 3 ml) and the samples were placed in a test tube from 2-18 hours at 37° C. with sonication. It was found that sonication is not a necessary requirement and the times of soaking vary with concentration of hyaluronidase and trypsin and can be as little as 2 hours. The above method of soaking has been previously used on human tissue and is set forth in the Journal of Rheumatology, 12:4, 1985 by Gust Verbruggen et al titled Repair Function in Organ Cultured Human Cartilage Replacement of Enzymatically Removed Proteoglycans During Longterm Organ Culture. After repeated washes with sterile DI water, the hydrated plug samples and cartilage were frozen at −70° C. and lyophilized to reduce water content within a range of about 0.1% to about 8.0%. In an alternative usage, the plug samples and cartilage were frozen after processing.

The osteochondral plug 20 which has been treated as noted above is placed in a bore or core 60 which has been cut in the lesion area of the bone 100 of a patient with the upper surface of the cartilage cap 24 being proud or substantially flush with the surface of the cartilage 102 remaining at the area being treated. The length of the osteochondral plug 20 is preferably the same as the depth of the bore 60 so that the base of the plug implant is supported and the articular cartilage cap 24 is level with the articular cartilage 102. With such load bearing support the graft surface is not damaged by excess weight or bearing loads known to cause micromotion interfering with the graft interface producing fibrous tissue interfaces and subchondral cysts.

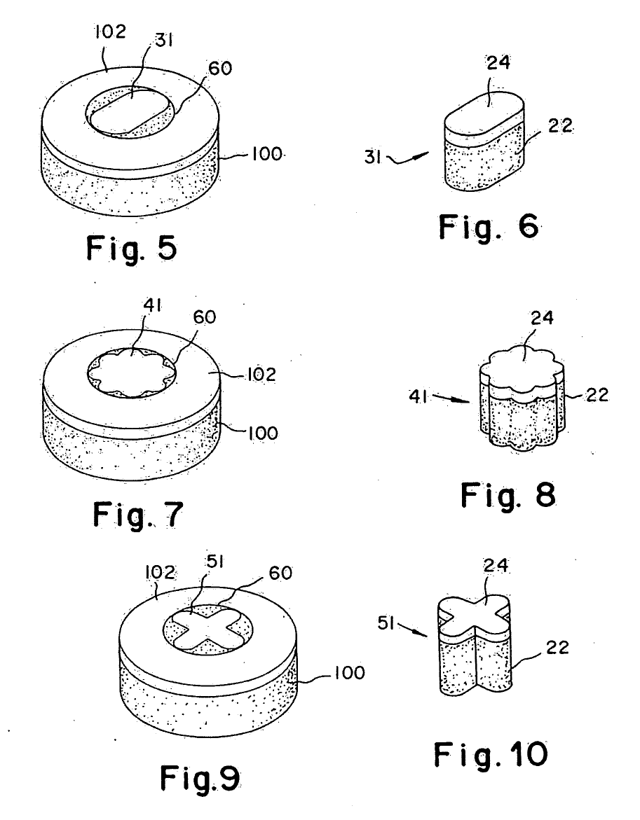

The plug 20 is movable within bore 60 while resting on the base of the bore 60 and if centered in the bore 60 does not touch the side walls of the bore or if touching does not have an interference fit. The osteochondral plug 20 which is referred to as a plug is also envisioned in various shapes namely, a cylindrical shape 21 as shown in FIG. 4, an oval shape 31 as shown in FIGS. 5 and 6, a scalloped shape 41 as shown in FIGS. 7 and 8 and a cruciate shape 51 as shown in FIGS. 9 and 10.

The remainder of the implant area is filled with a milled or minced cartilage mixture 30 having a size generally less than 1 mm of putty or gel together with a biological carrier and one or more of the following additives. The additives are one or more of chondrogenic stimulating factors including, but not limited to growth factors (FGF-2, FGF-5; 1GF-1, TGF-β, BMP-2, BMP-7, PDGF, VEGF), human allogenic or autologous chondrocytes, human allogenic cells, human allogenic or autologous bone marrow cells, human autologous and allogenic human stem cells, demineralized bone matrix, insulin, insulin-like growth factor-1, interleukin-1 receptor antagonist, hepatocyte growth factor, platelet-derived growth factor, Indian hedgehog and parathyroid hormone-related peptide.

If desired, demineralized or partially demineralized bone powder having a size range from 200 to 850 microns with a weight ranging from 1% to 35% of the cartilage mixture can be added to the milled cartilage mixture 30.

Suitable organic glue material can be used to keep the implant fixed in place (centered) or positioned as desired in the implant area Suitable organic glue material can be found commercially, such as for example TISSEEL® or TISSUCOL® (fibrin based adhesive; Immuno AG, Austria), Adhesive Protein (Sigma Chemical, USA), and Dow Corning Medical Adhesive B (Dow Corning, USA), fibrinogen, thrombin, elastin, collagen, casein, albumin, keratin and the like.

### EXAMPLE 1

A non-viable or decellularized osteochondral plug consisting of a subchondral bone base and overlying cartilage cap is treated with a solution or variety of solutions to remove the cellular debris as well as the proteoglycans as noted in the treatment described above. It is believed that this removal provides signaling to stimulate the surrounding chondrocytes and also the host's bone marrow and other mesenchymal stem cells to migrate into the graft to proliferate and form new proteoglycans and other factors producing new matrix. The diameter or diagonal of the plug ranges from 1 mm to 30 mm but is preferably 4 mm to 10 mm which is small enough to fit through the endoscopic cannula, but large enough to minimize the number of plugs needed to fill large defects. This size provides good results at the recipient site and provides a more confluent hyaline surface. The thickness of subchondral bone can be modified to match the anatomy of the patient so that the surface cartilage of the plug will be even with and follow the surface cartilage of the host tissue. The treated plug also creates a more porous matrix, which allows more cells to enter. This plug and minced hyaline cartilage can be stored frozen or freeze dried and support any of the mentioned chondrogenic stimulating factors. The plug can be inserted arthroscopically similar to the mosaicplasty procedure or through an open incision. The plug can be made in various dimensions depending on the size of the defect being treated.

This design uses the allograft cartilage putty or gel as a biological glue in a prepackaged amount to hold the osteochondral plug in place and to fill the space between the plugs in larger defects that require more than one plug. The putty or gel enhances the tissue integration between the plug and host tissue. Preferably, the plug has a smaller diameter or cross section than the bore of the debrided cartilage defect. The milled or minced cartilage putty or gel is injected into the defect after the plug or plugs are inserted or can be injected before insertion of the plug(s). The putty or gel fills the space between the plug and the sides of the defect. Thus, the plug or plugs initially are moveable in the defect bore area. For larger defects requiring more than one plug, the putty or gel also fills the space between the plugs. The term putty and paste denote a less flowable mixture and are used interchangeably.

The operation of placing a preshaped allograft implant assembly in a cartilage defect utilizes a subchondral bone and an overlying cartilage cap plus which has been treated to remove cellular debris and proteoglycans and milled cartilage in a carrier. The steps of the operation are: (a) drilling a hole which can be in the form of a cylindrical bore in a patient at a site of a cartilage defect, a depth which equal to the length of the bone and cartilage cap plug implant, (b) placing a preshaped osteochondral plug having a cross section which is less than the cross sectional area of the cylindrical bore with a length which is equal to or slightly greater than the depth of the bore allowing the structure to be moveable within said bore in the cylindrical hole; and (c) placing a mixture of milled cartilage in a bioabsorbable carrier in the drilled cylindrical hole around the preshaped osteochondral plug. Alternately the plug may be fixed in position in the cylindrical hole through the use of a biological glue.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What we claim is:

1. A cartilage repair assembly comprising a sterile shaped structure of subchondral bone with an integral overlying cartilage cap, said shaped structure being dimensioned to fit in a drilled bore in a cartilage defect area so that said shaped bone and cartilage cap when centered in the bore does not engage the side wall of the bore in an interference fit, said shaped structure being treated to remove cellular debris and proteoglycans, and sterile milled cartilage pieces mixed in a carrier surrounding said bone plug in said bore.

2. A cartilage repair assembly as claimed in claim 1 wherein said milled cartilage pieces are sized less than 1 mm.

3. A cartilage repair assembly as claimed in claim 1 wherein said cartilage is allograft cartilage.

4. A cartilage repair assembly as claimed in claim 1 wherein said cartilage is autologous cartilage.

5. A cartilage repair assembly as claimed in claim 1 wherein said shaped structure has a shape taken from a group consisting of a cylinder, an oval, a cruciate, and scallop.

6. A cartilage repair assembly as claimed in claim 1 wherein said milled cartilage pieces and carrier includes an additive taken from one or more of a group consisting of growth factors, human allogenic cells, human bone autologous marrow cells, human allogenic bone marrow cells, stem cells, demineralized bone matrix, cartilage, and insulin.

7. A cartilage repair assembly as claimed in claim 6 wherein said demineralized bone matrix comprises bone powder having a size ranging from 200 to 850 microns and a weight ranging from 1% to 35% of the cartilage mixture.

8. A cartilage repair assembly as claimed in claim 1 wherein said carrier includes a bioabsorbable carrier consisting of one or more of a group consisting of sodium hyaluronate, gelatin, collagen, chitosan, alginate, buffered PBS, Dextran or polymers.

9. A cartilage repair assembly as claimed in claim 1 wherein said milled cartilage is hyaline cartilage.

10. A cartilage repair assembly as claimed in claim 1 wherein said milled cartilage is fibrocartilage.

11. A cartilage repair assembly as claimed in claim 1 wherein said milled cartilage is a mixture of fibrocartilage and hyaline cartilage.
